(19) United States
(12) Patent Application Publication
(10) Pub. No.: US 2004/0208928 A1
(43) Pub. Date: Oct. 21, 2004

(54) Method for preparing an orally administrable formulation for controlled release

(75) Inventors: Chao-Wei Liao, Miaoli Hsien (TW); Peggy Lin, Miaoli Hsien (TW); Chung-Nan Weng, Miaoli Hsien (TW)

Correspondence Address: Ladas & Parry, 26 West 61st Street, New York, NY, 10023, US

(73) Assignee: ANIMAL TECHNOLOGY INSTITUTE TAIWAN

(21) Appl. No.: 10/414,177

(22) Filed: Apr. 15, 2003

(51) Int. Cl. A61K 9/22; A61K 9/16; A61K 9/50

(52) U.S. Cl. 424/468; 264/4

(57) ABSTRACT

The present invention provides a method for preparing an orally administrable formulation comprising a biologically active ingredient for the controlled release in a neutral or basic environment, which method comprises the steps of: (a) dispersing powder ethylcellulose with an average diameter of from about 0.1 μm to about 300 μm in an aqueous dispersion to provide an enteric encapsulant; (b) mixing the biologically active ingredient and the enteric encapsulant obtained in step (a) to obtain a mixture; and (c) spray-drying the mixture obtained in step (b) for about 10 sec. to 15 sec. in a drying chamber at a chamber temperature of about 45° C. to about 80° C. to obtain an orally administrable formulation. The orally administrative formulation prepared by the method of the invention is also provided.

### BACKGROUND OF THE INVENTION

1. Field of the Invention

The invention mainly relates to a method for preparing an orally administrable formulation for controlled release of a biologically active ingredient.

2. Description of the Related Art

Biologicals (biological composition) such as microorganisms, enzymes and proteins, are used as vaccines, and heat liable pharmaceutical compositions for humans and animals. Generally, biologicals are usually delivered by injection to maintain a good biological activity. However, the costs of injection and the resistance of the subjects to be administrated limit the applications of the biologicals.

To overcome the limitations of injection administration, orally administrable formulations are developed. However, it is difficult and complex to prepare an effective formulation for an oral administration of a biological. For instance, biologicals administrated orally are absorbed and targeted or reacted in the intestines. In other words, a biological should be encapsulated with an enteric material for delivering and controlling release in the intestines to avoid acid damage in the stomach. Therefore, the encapsulation process of heat-liable biological is normally conducted by a coating process. The coating process, either with an organic solvent or an aqueous system, has been applied extensively in pharmaceutical systems. The impact of various external and internal forces against solvent systems, such as the risk of explosive hazard and environmental pollution, has revitalized an interest in aqueous coating alternatives. However, using water as a solvent, more time or energy is required for evaporation. In conventional preparation for a pre-oral enteric formulation containing a biological, the material suitable for an enteric coating is used, such as cellulose acetate phthalate (CAP), methyl methacrylate methacrylic acid copolymer, hydroxy propyl methyl cellulosephthalate (HPMCP), and polyvinyl acetate phthalate (PVAP). Such macromolecular materials are dissolved in a neutral or basic environment to release the biologically active ingredient encapsulated therein, but not in an acid environment. However, the encapsulation process must be conducted at a high temperature or harsh organic solvent, under which the biological activity of the biologically active ingredient is decreased dramatically. Besides, the costs for coating process are very high, especially for animal use.

Polymeric latex dispersed in an aqueous solution was found to be used an encapsulant because of hydrophobic and latex film forming properties. Since such polymeric latex made water in the solution evaporated at a quite low energy, it can be used for encapsulation of a biologically active ingredient under a low temperature (Kulvanich, P. and Leesawat, P. 1996, Release characteristics of the matrices prepared from co-spray dried theophylline and ethylcellulose with/without channeling agents. Proceedings of the International Symposium on Controlled Release of Bioactive Materials, 23, 143-144). Latex together with ethylcellulose aqueous polymeric dispersion (Aquacoat™) purchased from FMC Corporation (Philadelphia, USA) was used to prepare an enteric formulation containing a biologically active ingredient by a co-spray drying process. (Liao C. W. et al. 2001, Release characteristics of microspheres prepared by co-spray drying Actinobacillus pleuropneumoniae antigens and aqueous ethyl-cellulose dispersion, J. Microencapsulation, Vol. 18, NO. 3, 258-297)

Given the above, an economical, effective and orally administrative formulation of a biologically active ingredient is desired.

### SUMMARY OF THE INVENTION

It is surprisingly found that powder ethylcellulose, which is a known coating material, can be used for preparing an “enteric” encapsulant for a biologically active ingredient by a co-spray drying process. The invention provides an effective and economical process for preparing an orally administrative formulation for a biologically active ingredient.

One subject of the invention is to provide a method for preparing an orally administrable formulation comprising a biologically active ingredient for the controlled release in a neutral or basic environment, which method comprises the steps of:

(a) dispersing powder ethylcellulose with an average diameter of from about 0.1 μm to about 300 μm in an aqueous dispersion to provide an enteric encapsulant;

(b) mixing the biologically active ingredient and the enteric encapsulant obtained in step (a) to obtain a mixture; and

(c) spray-drying the mixture obtained in step (b) and retaining the mixture for about 10 sec. to 15 sec. in a drying chamber at a chamber temperature of about 45° C. to about 80° C. to obtain an orally administrable formulation.

Another subject of the invention is to provide an orally administrable formulation prepared by the method of the invention.

### BRIEF DESCRIPTION OF THE DRAWINGS

FIG. 1 illustrates the result of protein release assay of the orally administrable vaccines of Formulas K to P according to Example 1 of the invention.

FIG. 2 illustrates the result of protein release assay of the orally administrable vaccines of Formulas 1 to 9 according to Example 2 of the invention.

FIG. 3 illustrates the result of antibody amount in mice stimulated with orally administrable vaccines of Formulas L, N, and O according to Example 1 of the invention, and that with injected antigens; a: IgG; b: IgA. Values shown are means±standard deviations of the titres of 6 mice at each sampling. Any standard deviations not seen are too small to be visible above the symbols.

FIG. 4 illustrates the result of antibody amount in mice stimulated with orally administrable vaccines of Formulas 4 to 6 according to Example 2 of the invention, and that with injected antigens; a: IgG; b: IgA. Values shown are means±standard deviations of the titres of 6 mice at each sampling. Any standard deviations not seen are too small to be visible above the symbols.

### DETAILED DESCRIPTION OF THE INVENTION

The invention provides a method for preparing an orally administrable formulation comprising a biologically active ingredient for the controlled release in a neutral or basic environment, which method comprises the steps of:

(a) dispersing powder ethylcellulose with an average diameter of from about 0.1 μm to about 300 μm in an aqueous dispersion to provide an enteric encapsulant;

(b) mixing the biologically active ingredient and the enteric encapsulant obtained in step (a) to obtain a mixture; and

(c) spray-drying the mixture obtained in step (b) and retaining the mixture for about 10 sec. to 15 sec. in a drying chamber at a chamber temperature of about 45° C. to about 80° C. to obtain an orally administrable formulation.

As used herein, the term “biologically active ingredient” refers to a biological material or substance with a biological activity in humans or animals. Normally, the biologically active ingredient according to the invention is pH sensitive, and is absorbed in the intestine. The biologically active ingredient may be absorbed into the capillary in the intestines. The biologically active ingredient includes a microorganism, a protein, an enzyme, serum, and the mixture thereof. In a more preferred embodiment, the biologically active ingredient is a microorganism, which may be live. The live microorganism may also be pre-treated to lower the toxicity or increase the compatibility. The microorganism may be any microorganism which provides a biological activity, and may be inactivated by heat or a chemical, such as formaldehyde. In one preferred embodiment of the invention, the microorganism is selected from the group consisting of Escherichia coli, Lactobacillus acidophilus, Lactobacillus pentose, Bacillus subtilis, and the mixture thereof.

As used herein, the term “orally administrable formulation” refers to a composition for oral administration, which is suitable for. In one embodiment of the invention, the orally administrable formulation may be used as a vaccine or a pharmaceutical preparation or an oral probiotic supplementation. The term “vaccine” as used herein refers to an antigenic substance (such as an antigen) for antibody production, which provides an effect in protecting a subject from infection. Usually, vaccines are usually used for the prophylaxis of epidemic diseases in humans and animals. To maintain the biological activity of the biologically active ingredient, such as an antigen in a vaccine, the biologically active ingredient must be encapsulated with an enteric encapsulant to pass by the stomach but release it into the intestines. For example, the antigenic substrate released in the intestines induces the mucosa (such as Peyer's patch) to produce immunoglobulins to provide a preliminary protection from infections. Recently, many studies show that an oral administration is a safe, convenient and economical way to induce immune response. The orally administrable formulations may be added in feeds or fed to animals. It is the most convenient way to induce immune responses in animals for protection from infections.

The orally administrable formulation may be a solid or a liquid core embedded in a coating, and is in the form selected from the group consisting of a microcapsule, a microparticle, a microsphere, a micrometric or a microbead, a capsule containing microcapsules, and a table containing microcapsules.

According to the invention, the biologically active ingredient is encapsulated to form a microcapsule. In an embodiment of the invention, the microcapsule may be further embedded in a coating or a unit that is easier to be taken. Usually, the particles of the formulation have a diameter of from about 1 to 2,000 μm.

As used herein, the term “controlled release” refers to a condition that a biologically active ingredient is controlled to release in a particular environment. Preferably, the biologically active ingredient is controlled to release in a neutral or basic environment; more preferably, in an enteric environment. As used herein, the term “enteric environment” refers to the enteric cavity or a physiologically equivalent environment.

As used herein, the term “encapsulant” refers to a material for encapsulating or coating the biologically active ingredient. The encapsulant has a property of film forming to encapsulate or coat the biologically active ingredient. The encapsulant according to the invention comprises an aqueous ethylcellulose dispersion. In a preferred embodiment, the encapsulant is prepared by dispersing powder ethylcellulose with an average diameter of from 0.1 μm to 300 μm in an aqueous solution. More preferably, the average diameter of powder ethylcellulose in the dispersion is from 0.3 μm to 3 μm. In an embodiment of the invention, the dispersion of ethylcellulose has a viscosity ranging from about 5 to about 10⁵ cps; more preferably, from about 5 to about 24 cps; and most preferably, from about 18 to 24 cps. According to the invention, the encapsulant provides an effect in controlling the release of the biologically active ingredient in a neutral or basic environment; more preferably, in an enteric environment. According to the invention, the biologically active ingredient may be incorporated into an excipient, carrier, or adjuvant. In a preferred embodiment of the invention, the excipient is selected from the group consisting of milk powder, serum, talc, and the mixture thereof. It is surprised to find that such preferred excipient can provide an enteric effect. Furthermore, the encapsulant can protect the biologically active ingredient from degradation or damage at storage or in the stomach. In a preferred embodiment, the encapsulant further comprises a detergent to allow the ethylcellulose dispersion to form a film. More preferably, the detergent is selected from the group consisting of cetyl alcohol, sodium dodecyl sulfate (SDS), and the mixture thereof. In an embodiment of the invention, the encapsulant further comprises an enteric encapsulant, which allow the biologically active ingredient to be controlled to release in the intestines. Preferably, the enteric encapsulant is selected from the group consisting of cellulose acetate phthalate (CAP), methyl methacrylate methacrylic acid copolymer, hydroxy propyl methyl cellulosephthalate (HPMCP), polyvinyl acetate phthalate (PVAP), and the mixture thereof. According to the invention, a protectant may be further added into the formulation. In a preferred embodiment of the invention, the protectant is selected from the group consisting of glycerol, polyethylene glycol and the derivatives thereof, and the mixture thereof.

According to the invention, the step (a) is to provide an enteric encapsulant, which is prepared by dispersing powder ethylcellulose in an aqueous solution, such as water. According to the invention, the preparation of an encapsulant is very easy to perform at a low cost.

In the step (b) of the method, the biologically active ingredient and the enteric encapsulant are mixed to obtain a mixture. The biologically active ingredient can be mixed directly or pretreated in accordance different purposes. For instance, a co-spray drying process may be used for mixing the biologically active ingredient and the encapsulant. In an embodiment of the invention, the biologically active ingredient is granulated to form a core before encapsulation to prepare a formulation with a large particle size.

In the step (c) of the method, the mixture obtained in step (b) is spray dried for about 10 sec to 15 sec in a drying chamber at a chamber temperature of about 45° C. to about 80° C. to obtain the orally administrable formulation. According to the invention, the temperature is not too high, and therefore, the biological activity of the biologically active ingredient can be maintained. According to the invention, the chamber temperature varies with the kind of the biologically active ingredient used. In a preferred embodiment, the chamber temperature ranges from about 60° C. to about 65° C. In an embodiment of the invention, the mixture may be spun and spray dried by inletting hot air at a temperature of about 50° C. to about 200° C. for maintaining the chamber temperature. Because the mixture contacts with the hot air in a very short time and the water contained in the formulation absorbs latent heat to gasify, the mixture can be dried at a low temperature. Preferably, the mixture is spun at the speed rate of about 10,000 rpm to about 40,000 rpm.

Optionally, the process according to the invention further comprises a step (d) where the orally administrable formulation in step (c) is collected at a temperature of about 15° C. to about 45° C. in an out-let collecting tank.

In one embodiment of the invention, the method may be carried out in a spray dryer, which comprises (a) a heater for heating air; (b) an atomizer for atomizing the mixture to form micro particles; (c) a drying chamber where the wet micro particles contact the hot air to evaporate water in the micro particles and to form dry powder; (d) a cyclone separator for collecting the powder; and (e) a fan for drafting and exhausting air.

The orally administrable formulation prepared by the method according to the invention have the advantages of: (1) providing a resistance to gastric juice; (2) having the ability to control to release or slow release in the enteric environment; (3) having a good compatibility with the biologically active ingredient and additive; (4) having a stability; (5) forming a continuous film and a capsule after drying; (6) being non-toxic and safe; (7) being at a low cost; and (8) being suitable for applying in granulating and drying. Furthermore, the biologically active ingredient of the formulation according to the invention has a good biological activity.

The following Examples are given for the purpose of illustration only and are not intended to limit the scope of the present invention.

### EXAMPLE 1

### Orally Administrable Escherichia coli Vaccine Encapsulated by Ethylcellulose Powder Dispersion

Encapsulant comprising ethylcellulose: Ethylcellulose powder (15% to 90%) was dispersed in water with the addition of 0.5 g to 2 g sodium dodecyl sulfate (SDS) to form a dispersion. The dispersion was added with 0.5 g to 3 g melted cetyl alcohol with vigorous agitation to form the encapsulant comprising ethylcellulose.

Inactivated bacterial culture: The inactivated bacterial culture was prepared by culturing and inactivating Escherichia coli F18-VTST with formaldehyde. A single colony of E. coli F18-VTST was selected and inoculated in 50 mL of LB broth containing 10 ppm to 100 ppm of streptomycin, kanamycin, ampicilline, or tracycline or 0.2% to 2% of sorbitol at 37° C. with shaking overnight. Twenty mL of the culture was transferred into 400 mL of the same medium and incubated for two days without shaking. Formaldehyde (0.5% to 3%) was added and shaken at 37° C., and then stayed at 4° C. to 10° C. for one day.

Co-spray drying: Ten % to 82% of the inactivated E. coli culture, 0% to 52% of water, 1% to 5% of porcine serum, and 1% to 2% glycerol were mixed and added with 16% to 88% of the encapsulant comprising ethylcellulose. After mixing well, 0.1% to 2% talc was then added and mixed. The mixture was transferred to a spray dryer in a condition of a dry chamber temperature of 45° C. to 80° C., a hot air temperature of 120° C. to 200° C., a tank temperature of 60° C. to 65° C., and a inletting temperature of 40° C. to 50° C. and spun at 10,000 rpm to 40,000 rpm for drying for 10 sec. to 15 sec. After micro-granulation and evaporation, the microencapsule was obtained and collected by a cyclone separator. The orally administrable vaccine was stored at 4° C.

The formulations to be tested were listed in Table 1, wherein “HPMCP” stood for hydroxy propyl methyl cellulosephthalate; ECN7-A was ethylcellulose purchased from Hercules Incorporated Aqualon Division with the viscosity of 5.6 to 8 cps; ECN7 was ECN7-A milled to average particle size of 5 μm to 10 μm; ECN22-A was ethylcellulose purchased from Hercules Incorporated Aqualon Division with the viscosity of 18 to 24 cps; ECN22 was ECN22-A milled to average particle size of 5 μm to 10 μm; and Aquacoat™ stood for ethylcellulose aqueous polymeric dispersion purchased from FMC Corporation.

TABLE 1

| | | Bac- | | | |
|---|---|---|---|---|---|
| For- | | terial | | | Porcine |
| mula | Encapsulant | culture | HPMCP | Talc | serum |
| K | ethylcellulose-ECN7-A: 30 g | 500 mL | 5 g | 2.5 g | 10 g |
| | dd H₂O: 100 g | | | | |
| | SDS: 1.5 g | | | | |
| | Cetyl alcohol: 1.5 g | | | | |
| L | ethylcellulose-ECN7: 30 g | 500 mL | 5 g | 2.5 g | 10 g |
| | dd H₂O: 100 g | | | | |
| | SDS: 1.5 g | | | | |
| | Cetyl alcohol: 1.5 g | | | | |
| M | ethylcellulose-ECN22-A: 30 g | 500 mL | 5 g | 2.5 g | 10 g |
| | dd H₂O: 100 g | | | | |
| | SDS: 1.5 g | | | | |
| | Cetyl alcohol: 1.5 g | | | | |
| N | ethylcellulose-ECN22: 30 g | 500 mL | 5 g | 2.5 g | 10 g |
| | dd H₂O: 100 g | | | | |
| | SDS: 1.5 g | | | | |
| | Cetyl alcohol: 1.5 g | | | | |
| O | Aquacoat ™: 100 g | 500 mL | 5 g | 2.5 g | 10 g |
| P | — | 500 mL | 35 g | 2.5 g | 10 g |

The vaccine was stored at 4° C. The efficiency assay and protein release assay were conducted after one week, and the stability was monitored by observing the features and particle sizes. The efficiency assay and protein release assay were further conducted after 1, 2, 3, 6, and 12 months, respectively.

Efficiency assay: Bacteria with total protein weighted to 2.5 g were used for encapsulating as described above, and subjected to the assays. Because the antigen amount in the bacteria was low and in a particular ratio to total protein, the amount of total protein was used as an indicator in the assay. One hundred mg of the powdered vaccine was placed in a 10-mL container and added with 1 mL of 10% of SDS with shaking for 1 hour. One mL of trichloromethane and acetone was then added and deposited in a laminar flow for drying overnight. After drying, 10 mL of PBS was added and mixed. One and a half mL of a sample was then taken and centrifuged at 12,500 rpm for 10 min. For each sample, the protein in the supernatant was quantified using Bio Rad™ protein assay reagent. The values of the protein estimated by the assay dividing the 2.5 g total protein was taken for evaluating the encapsulating efficiency and shown in Table 2. In view of the data of Table 2, the efficacy was very high, around 94-96%.

TABLE 2

| | Assay content of protein in | Recovery from 25 g original |
|---|---|---|
| Sample No. | microsphere samples (g) | protein (%) |
| 1 | 23.8 ± 0.7 | 95.2 |
| 2 | 23.5 ± 0.6 | 94.0 |
| 3 | 24.0 ± 0.4 | 96.0 |

Protein release assay: The process of the assay was described by Liao et al. (2001) at 37° C. and 100 rpm. Two hundred mg vaccine with total protein weighted to 15 mg was first reacted in 500 mL of 0.01 N HCl for 2 hours (pH 2), and then added with 0.2 M tri-basic sodium phosphate until the pH value of 6.8 for further reaction. One and a half mL of a sample was taken every 0.5-hour or every 1 hour during the assay and centrifuged at 15,000 g, and the supernatant was taken stored at −20° C. The supernatant was then diluted in PBS buffer and quantified with Bio Rad™ protein assay reagent. BSA standard solution was also taken for estimating the protein amount.

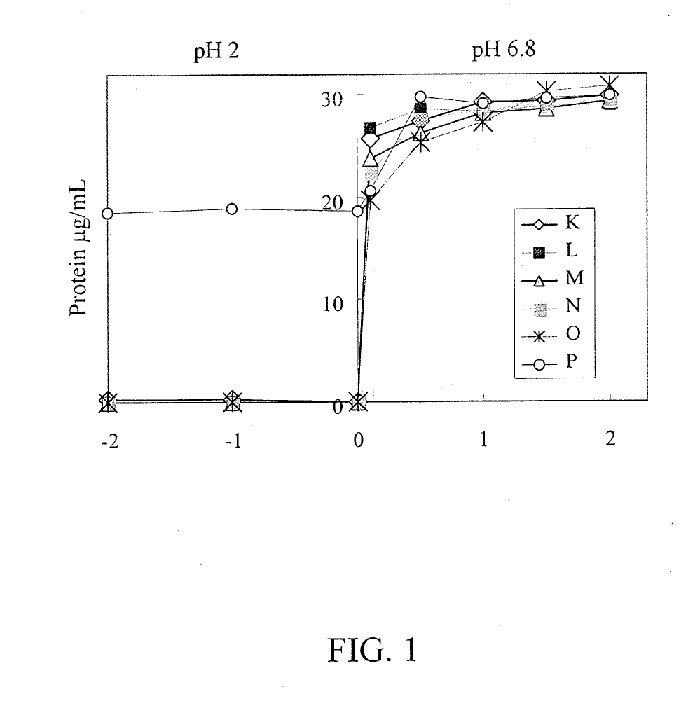

The results of the assay were shown in FIG. 1. Formula P without ethylcellulose did not have a good controlled release property in the intestines (pH 6.8) even in the assistance of HPMCP. The result of Formula O prepared from Aquacoat™ purchased from FMC Corporation show that Aquacoat™ can be used for preparing a good orally administrable vaccine. Furthermore, Ethylcellulose of EC-N7 (Formulas K and L) and EC-N 22 (Formulas M and N) can also provide a good effect in controlling the release of a bacterium in the intestines. Furthermore, no precipitates were found in the formulation prepared from the encapsulant comprising Formula N, and block tubes. The release rate of Formula N at pH 6.8 was fast in the first half hour, and Formula N provides an effect in controlling the release in the intestines. However, the release rate of Formula L at pH 6.8 was too fast and might lead to crash of the film.

### EXAMPLE 2

### Orally Administrable Escherichia coli Antigen Vaccine Encapsulated by Ethylcellulose Powder Dispersion

The preparation of encapsulant comprising ethylcellulose and the process of co-spray drying used herein are as described in Example 1.

The antigens of Escherichia coli: E. coli F18-VTST was used in this example. The bacterial stock was maintained at −70° C. in LB medium containing 50% glycerol. For preparation of the formalin-killed bacterial antigens, 1 ml inocula was incubated in 10 mL LB at 37° C. overnight, and transferred into 200 mL of LB in a 500 mL Hinton flask and cultured at 37° C. for 15 h. After the culture was lightly homogenized by a homogenizer (polytron pt-300, Kinematica AG), 0.5% formalin was added into the culture broth and mixed at 37° C. for 1 h and then incubated at 4° C. for 15 h. To assure that there were no viable bacteria in the broth, 5 ml of the inactivated broth was aspirated into 200 mL of LB in a 500 mL Hinton flask and cultured at 37° C., until there was no bacterial growth after 1-week cultivation. The formalin-inactivated broth was then used as an antigen for vaccine formulation.

The formulations were listed in Table 3, wherein “HPMCP” refers to hydroxy propyl methyl cellulosephthalate; N7, 14, 22, 55, and 100 were powder ethylcellulose purchased from Hercules Incorporated Aqualon Division with the viscosity of 7, 14, 22, 55, and 100 cps, respectively; Aquacoat™ refers to an ethylcellulose aqueous polymeric dispersion purchased from FMC Corporation.

TABLE 3

| | | | | | Porcine |
|---|---|---|---|---|---|
| Formula | Encapsulant | Antigen | HPMCP | Talc | serum |
| 1 | ethylcellulose-N7: 5 g | 100 mL | 1 g | 0.5 g | 0.5 g |
| | dd H₂O: 100 mL | | | | |
| | SDS: 0.5 g | | | | |
| | Cetyl alcohol: 0.5 g | | | | |
| 2 | ethylcellulose-N7: 10 g | 100 mL | 1 g | 0.5 g | 0.5 g |
| | dd H₂O: 200 mL | | | | |
| | SDS: 1 g | | | | |
| | Cetyl alcohol: 1 g | | | | |
| 3 | ethylcellulose-N7: 15 g | 100 mL | 1 g | 0.5 g | 0.5 g |
| | dd H₂O: 200 mL | | | | |
| | SDS: 1 g | | | | |
| | Cetyl alcohol: 1 g | | | | |
| 4 | ethylcellulose-N14: 10 g | 100 mL | 1 g | 0.5 g | 0.5 g |
| | dd H₂O: 100 mL | | | | |
| | SDS: 1.5 g | | | | |
| | Cetyl alcohol: 1.5 g | | | | |
| 5 | ethylcellulose-N22: 10 g | 100 mL | 1 g | 0.5 g | 0.5 g |
| | dd H₂O: 100 mL | | | | |
| | SDS: 1.5 g | | | | |
| | Cetyl alcohol: 1.5 g | | | | |
| 6 | ethylcellulose-N55: 10 g | 100 mL | 1 g | 0.5 g | 2 g |
| | dd H₂O: 100 mL | | | | |
| | SDS: 1.5 g | | | | |
| | Cetyl alcohol: 1.5 g | | | | |
| 7 | ethylcellulose-N100: 10 g | 100 mL | 1 g | 0.5 g | 2 g |
| | dd H₂O: 100 mL | | | | |
| | SDS: 1.5 g | | | | |
| | Cetyl alcohol: 1.5 g | | | | |
| 8 | dd H₂O: 100 mL | 100 mL | 11 g | 0.5 g | 2 g |
| | SDS: 1.5 g | | | | |
| | Cetyl alcohol: 1.5 g | | | | |
| 9 | Aquacoat ™: 30 mL | 100 mL | 1 g | 0.5 g | 2 g |

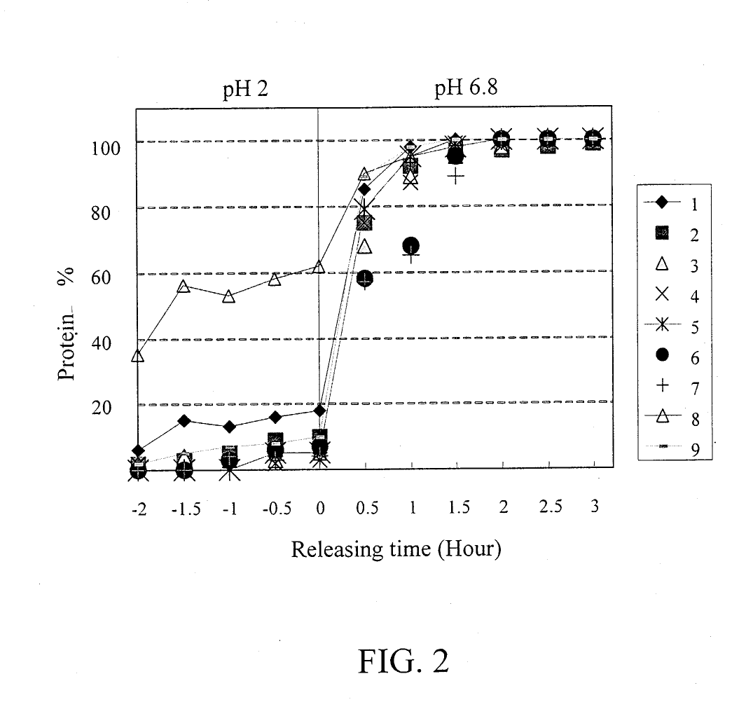

Protein release assay: The process of the assay was described in Example 1. The results of the assay were shown in FIG. 2. Each Formula except 8 had a good controlled release property in the intestines (pH 7). Formula 8 composed without ethylcellulose had no enteric characteristics, even added with 11 g HPMCP.

### EXAMPLE 3

### Live Bacterial Microencapsule for Preparation of a Probiotic Supplementation

Lactic acid bacteria (LAB), especially Lactobacillus and Bifidobacterium sp., are commonly present in the gastrointestinal tracts, and are effective in inhibiting pathogenic bacteria, antitumour and anticholesterolaemic activities, improving digestion, and stimulating the immune system. For instance, a supplement of LAB provides an effect in promotion of body weight gain, increase of feed conversion, enhancement of colonization of beneficial bacteria, and decrease of harmful intestinal bacteria in piglets.

Bacterial strains: Lactobacillus acidophilus and L. pentose were cultured for 3 days in MRS broth (Difco) at 37° C. and 5% CO₂, while Bacillus subtilis was cultured for 3 days in LB broth (Lennox, Difco) at 37° C. This SCP mixture contained L. acidophilus, L. pentose, and B. subtilis, at the final concentration of each bacterial count was adjusted to 10⁹ CFU/g using corn as the diluent.

Composition: Fresh cultures of L. acidophilus, L. pentose, and B. subtilis of 10⁷ to 1010 CFU/mL (80 to 90%) were mixed with milk powder (2 to 10%) and then about 10 to 20% ethylcellulose was added and mixed. After mixed, 0.1 to 2% of talc was then added and mixed. The mixture was transferred to a spray dryer in a condition of hot air temperature of 120° C. to 200° C., a tank temperature of 60° C. to 65° C., and a inletting temperature of 40° C. to 50° C. and spun at 10,000 rpm to 40,000 rpm. After micro granulation and water evaporation, the microencapsule with 10⁸ to 1011 CFU/g bacterial was obtained and collected by a cyclone separator. The composition was stored at 4° C.

The formulations of the vaccine were listed in Table 4, and wherein “bacteria” was the mixture of L. acidophilus, L. pentose, and B. subtilis; “HPMCP” stood for hydroxy propyl methyl cellulosephthalate; and ECN22 was ECN22-A milled to average particle size of 5 μm to 10 μm.

TABLE 4

| | | Milk | | |
|---|---|---|---|---|
| Formula | Bacteria | powder | HPMCP | Talc |
| Ethylcellulose-ECN22-A: 30 g | 1000 mL | 50 g | 10 g | 5 g |
| dd H₂O: 100 g | | | | |
| SDS: 1.5 g | | | | |
| Cetyl alcohol: 1.5 g | | | | |

Enumeration: Before drying, the organisms were enumerated on MRS agar for Lactobacillus and on LB agar for Bacillus. Each 0.1 g of spray-dried bacteria was rehydrated in the 9.9 mL of maximum recovery diluent (Oxoid). The cells were allowed to rehydrate for 2 hours, and then diluted further with diluent, and the suitable dilutions were plated as described above. The percent survival of bacteria was calculated as follows: % survival ═(N/N₀)×100, where N₀ is the number of bacteria per gram of dry matter before drying and N is the number of bacteria per gram of dry matter in the powder.

The results were shown in Table 5:

TABLE 5

| | | | | | Changes of |
|---|---|---|---|---|---|
| | Culture | | | Bacterial No./ | before and |
| | weight | | Microencapsule | Microencapsule | after |
| Bacteria | (kg) | Bacteria No. | weight (g) | weight (g) | encapsulating |
| L. acidophilus | 4 | 3.0 × 10⁸ | 595 | 2.0 × 10⁹ | 0.99 |
| B. subtilis | 4 | 5.2 × 10⁷ | 577 | 3.7 × 10⁸ | 1.02 |

Growth inhibition of enterpathogens: The interference of lactobacilli with the growth of harmful bacterial strains was evaluated by co-incubating E. coli O157:H7 and S. typhimurium individually with Lactobacillus strain. For each experiment, a tube containing 5 ml of MRS-LB (Luria-Bertani broth) broth (1:1) was inoculated with cell numbers (ca. 5×10⁵ CFU/ml) in the ratio 1:1 for Lactobacillus and the enteropathogen strain.

The results of the antagonistic activity of Lactobacillus against E. coli or S. typhimurium were shown in Table 6, demonstrating that the live bacterial microencapsule can inhibit harmful bacteria in the intestines.

TABLE 6

| | Lactobacillus | E. coli O157:H7 | Salmonella |
|---|---|---|---|
| Time | (log CFU/ml) | (log CFU/ml) | (log CFU/ml) |
| 24 hour | 10 | 4.2 | 5.1 |
| 48 hour | 7.8 | <2 | <2 |

### EXAMPLE 4

### Effect of Orally Administrable Escherichia coli Vaccine Encapsulated by Ethylcellulose Power Dispersion

Immunization in mice model: Six-week old female Balb/c mice were obtained, quarantined for 1 week prior to study, and maintained throughout the study on libitum with pelleted food and water throughout the experiment. The animals were randomly assigned to groups of six, which received combinations of oral or subcutaneous dosing. Antigens for inoculating the mice were prepared according to Table 7.

TABLE 7

| Group | High dose | Low dose |
|---|---|---|
| Formula N | 39.5 mg/0.5 mL bacterial | 12 mg/0.15 mL bacterial |
| | culture broth | culture broth |
| Formula O | 40.5 mg/0.5 mL bacterial | 13.5 mg/0.15 mL bacterial |
| | culture broth | culture broth |
| Injection | Normal dose (0.1 mL bacterial | — |
| | culture broth) | |
| Blank | 0.1 mL normal saline | — |

The oral vaccines were suspended in 0.2% acetic acid and administrated to the stomach of the mice via a blunt-tipped feeding needle at intervals of 10 days. Two weeks after the twice immunizations, blood was collected by the puncture of retroorbital plexuses. The positive control group was immunized with a subcutaneous injection of the 0.5 mL vaccines containing 0.25 mL of formalin inactive broth at intervals of 10 days for a total of three immunizations, and blood was also collected by the puncture of retroorbital plexuses after two weeks after the twice immunizations. Serum was obtained from coagulation at 4° C. for 12 h and centrifugation. Intestine larvage samples were collected by instilling 1 ml of washing buffer (PBS containing 100 μg/ml soybean trypsin inhibitor, 50 mM EDTA, 1 mM PMSF, 0.5% gelatin, and 0.05% NaN₃) into the intestine. The larvage was collected and stored at −20° C.

Measurement of antibody responses by an Enzyme-linked Immunosorbent Assay: The detection of antibodies was conducted by ELISA. Freshly cultured bacteria (OD 600=1.8) were treated with 0.1% triton-X 100, and the total protein amount was estimated. One hundred μg of protein was diluted with PBS as antigens. Each well of a 96-well plate was coated with 100 μL of antigens for first coating. A blood sample diluted in PBS was added into a well and reacted at 37° C. for one hour. HRP (Horse radish Peroxidase) covalent bonded goat anti-mouse IgG or IgA was then added and colorized by adding TMB/E. The absorption was measured at 620 nm. A standard curve with dilutions of anti-mouse IgG or IgA was constructed on each ELISA plate. The concentration of antibody in the sample was then determined.

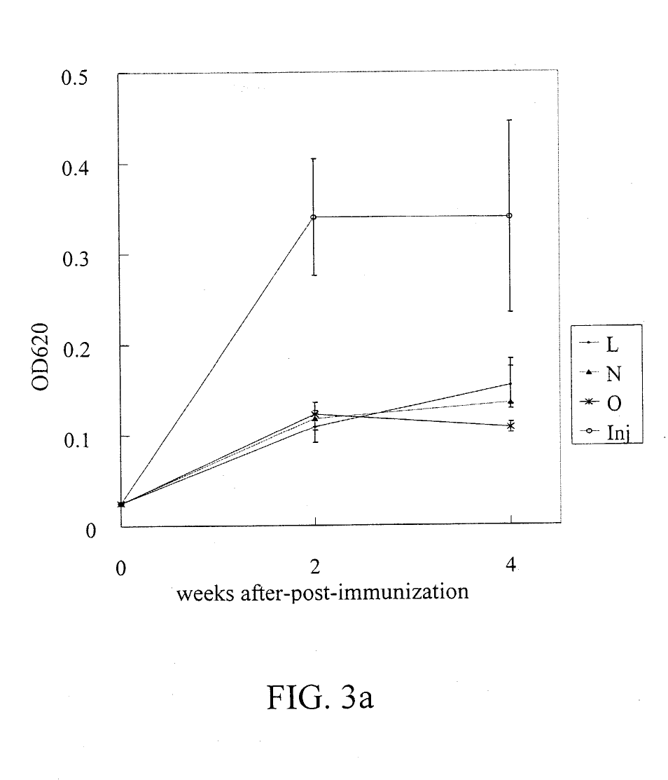

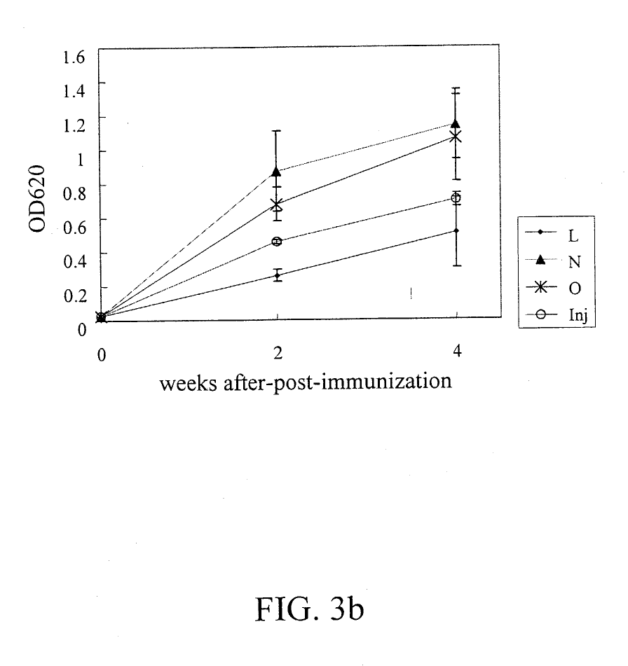

The result of ELISA was shown in FIG. 3. The secretion of IgG was raised enormously by injecting the antigens, and the effects of the orally administrable vaccine were similar. The results show that IgA was stimulated and secreted by the orally administrable vaccines of the invention, particularly Formulas N and O. That is, the vaccine according to the invention could stimulate immunity in mice.

Challenge in mice model: Five 4 to 5-week old female Balb/c mice were raised in an environment with air-condition and supplied with feed (laboratory rodent diet™ #5001) and clean water in the assay. Vaccines of Formula N and O, injecting vaccine and blank for inoculating the mice were prepared according to Table 7. The challenge was conducted on the fourteenth day after immunization. The estimation of lethal rate of mice and histopathological assay were conducted after one week. The result was shown in Table 8.

TABLE 8

| | Group | | | | |
|---|---|---|---|---|---|
| | 0 | 1 | 3 | 4 | 5 |
| Dilution of | Culture | 10-fold | 100-fold | 1,000-fold | 10,000-fold |
| bacteria | broth 0.1 mL | dilution 0.1 mL | dilution 0.1 mL | dilution 0.1 mL | dilution 0.1 mL |
| Mouse No. | 5 | 5 | 5 | 5 | 5 |

Death No.

| | | | | | |
|---|---|---|---|---|---|
| Formula N-H | 0 | 0 | 0 | 0 | 0 |
| Formula N-L | 3 | 3 | 2 | 0 | 0 |
| Formula O-H | 2 | 2 | 0 | 0 | 0 |
| Formula O-L | 4 | 4 | 1 | 0 | 0 |
| Injection | 4 | 4 | 0 | 0 | 0 |
| Blank | 5 | 5 | 4 | 1 | 0 |

The orally administrable vaccine of Formula N at a high dose could efficiently protect the mice from bacteria infection, and no mice died. Comparing the groups of Formula N and Formula O at a high dose with the control group, the protective effect of the vaccine was significant. The death number of mice in other groups in this assay varied upon the increase of the concentration of bacteria. As shown above, the orally administrable vaccine of Formula N at a high dose provide a protection from infections in animals, and the growth of harmful microorganisms was inhibited in the intestines. The formulation of the invention provides a safe, convenient and economical vaccine.

### EXAMPLE 5

### Effect of Orally Administrable Escherichia coli Antigen Vaccine Encapsulated by Ethylcellulose Powder Dispersion

The methods of immunization in mice model and measurement of antibody responses of orally administrable E. coli antigen vaccine encapsulated by ethylcellulose according to Example 2 were described in Example 4.

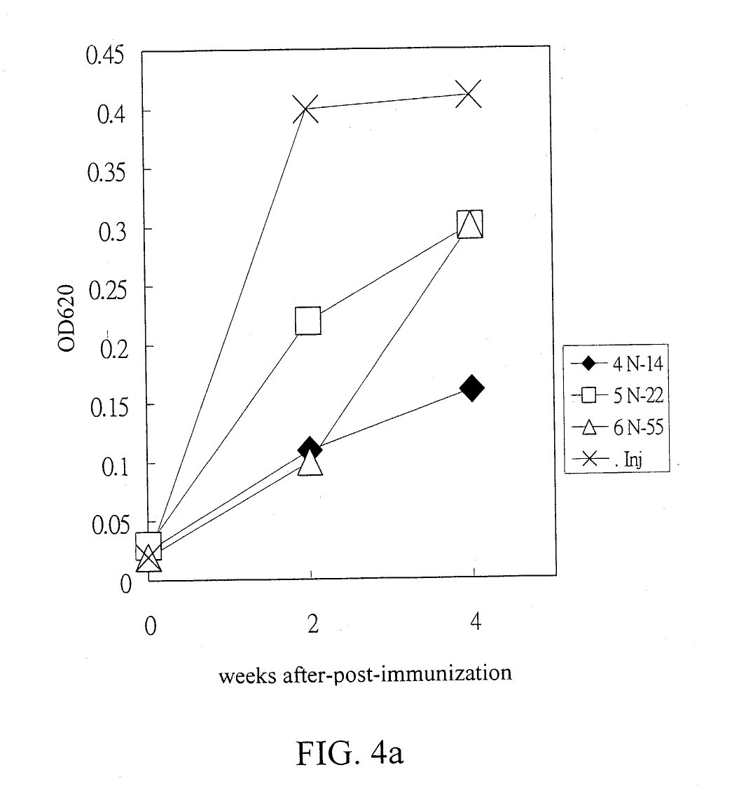

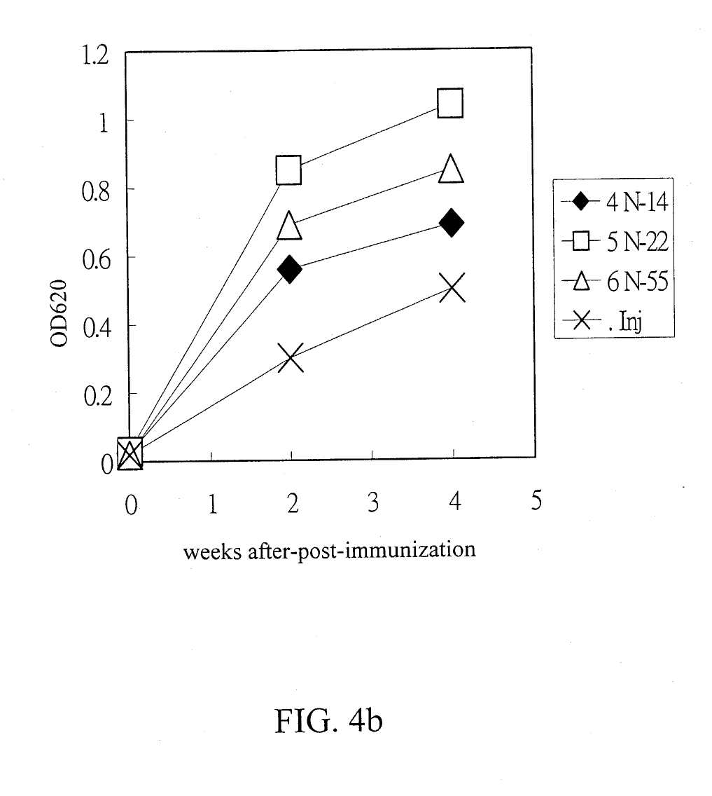

The ELISA result of Formulas 4 to 6 was shown in FIG. 4. The secretion of IgG was raised enormously by injecting the antigens, and the effects of the orally administrable vaccine were similar. The results show that IgA was stimulated and secreted by the orally administrable vaccines of the invention. That is, the vaccine according to the invention could stimulate immunity in mice.

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

What is claimed is:

1. A method for preparing an orally administrable formulation comprising a biologically active ingredient for the controlled release in a neutral or basic environment, which method comprises the steps of:
(a) dispersing powder ethylcellulose with an average diameter of from about 0.1 μm to about 300 μm in an aqueous dispersion to provide an enteric encapsulant;
(b) mixing the biologically active ingredient and the enteric encapsulant obtained in step (a) to obtain a mixture; and
(c) spray-drying the mixture obtained in step (b) for about 10 sec. to 15 sec. in a drying chamber at a chamber temperature of about 45° C. to about 80° C. to obtain an orally administrable formulation.

2. The method according to claim 1, wherein the powder ethylcellulose in the dispersion has a viscosity ranging from about 5 to about 10⁵ cps.

3. The method according to claim 1, wherein the powder ethylcellulose in the dispersion has a viscosity ranging from about 5 to about 24 cps.

4. The method according to claim 1, wherein the powder ethylcellulose in the dispersion has a viscosity ranging from about 18 to about 24 cps.

5. The method according to claim 1, wherein the powder ethylcellulose in the dispersion has an average diameter from 0.3 μm to 3 μm.

6. The method according to claim 1, wherein a detergent is further added to the aqueous ethylcellulose dispersion to obtain the encapsulant.

7. The method according to claim 6, wherein the detergent is selected from the group consisting of cetyl alcohol, sodium dodecyl sulfate (SDS), and the mixture thereof.

8. The method according to claim 1, wherein an enteric encapsulant is further added to the aqueous ethylcellulose dispersion to obtain the enteric encapsulant.

9. The method according to claim 8, wherein the enteric encapsulant is selected from the group consisting of cellulose acetate phthalate (CAP), methyl methacrylate methacrylic acid copolymer, hydroxy propyl methyl cellulosephthalate (HPMCP), polyvinyl acetate phthalate (PVAP), and the mixture thereof.

10. The method according to claim 1 wherein the biologically active ingredient is incorporated into a carrier, adjuvant or excipient.

11. The method according to claim 1 wherein a protectant is further added into the orally administrative formulation.

12. The method according to claim 10, wherein the excipient is selected from the group consisting of milk powder, serum, talc, and the mixture thereof.

13. The method according to claim 11, wherein the protectant is selected from the group consisting of glycerol, polyethylene glycol and the derivatives thereof, and the mixture thereof.

14. The method according to claim 1, wherein the biologically active ingredient is selected from the group consisting of a microorganism, a protein, an enzyme, a serum, and the mixture thereof.

15. The method according to claim 14, wherein the microorganism is selected from the group consisting of Escherichia coli, Lactobacillus acidophilus, Lactobacillus pentose, Bacillus subtilis, and the mixture thereof.

16. The method according to claim 14, wherein the microorganisms are live or inactive.

17. The method according to claim 1, wherein the orally administrative formulation is a vaccine.

18. The method according to claim 1 wherein the aqueous solution is water.

19. The method according to claim 1, wherein the biologically active ingredient in step (b) is granulated.

20. The method according to claim 1, wherein the chamber temperature in step (c) is from about 60° C. to about 65° C.

21. The method according to claim 1, wherein the mixture is spray dried by further spinning the mixture at the speed rate of about 10,000 rpm to about 40,000 rpm.

22. The method according to claim 1, wherein the mixture is spray dried by inletting hot air at a temperature of from about 50° C. to about 200° C.

23. The method according to claim 1 further comprising a step (d) where the orally administrable formulation in step (c) is collected at a temperature of about 15° C. to about 45° C. in an out-let collecting tank.

24. The orally administrative formulation comprising a biologically active ingredient prepared by the method according to any one of claim 1 to 23.

25. The formulation according to claim 24, wherein the biologically active ingredient is controlled to release in an enteric environment.

26. The formulation according to claim 24, which is in the form selected from the group consisting of a microcapsule, a microparticle, a microsphere, a micromatrice or a microbead, a capsule containing microcapsules, and a table containing microcapsules.
